(19) United States
(12) Patent Application Publication
(10) Pub. No.: US 2006/0251703 A1
(43) Pub. Date: Nov. 9, 2006

(54) Composition comprising an edible acid or its acidic salt and the use thereof

(76) Inventor: Shin-Jen Shiao, Hsinchu (TW)

Correspondence Address: Chauncey Johnson, 14625 Baltimore Avenue # 282, Laurel, MD, 20707, US

(21) Appl. No.: 10/554,315

(22) PCT Filed: Apr. 26, 2004
(86) PCT No.: PCT/CN04/00402

§ 371 Date: Oct. 24, 2005

(30) Foreign Application Priority Data
Apr. 24, 2003 (WO) PCT/CN03/00299

(51) Int. Cl. A61K 31/19; A61K 33/42; A61K 47/00

(52) U.S. Cl. 424/439; 424/601; 514/574

(57) ABSTRACT

This invention relates to a drug containing edible acid and/or acidic salt as active agent to treat and alleviate hypersensitivity diseases by lowering the humor pH and method; to improve individual hypersensitivity diseases by uses of health care food, which are made from said edible acid and/or acidic salt, or the acidic fruits containing thereof, or their products; foods lowering the risk of hypersensitivity and their preparation methods; drug to lower the humor acidity and to treat or alleviate disease caused by insects bite toxin; that also to be drug for cold or virus infection; drug for inflammation; drug for analgesic; and drug for thrombus disease; method lowers the hypersensitivity risk of clothing or grove and their products.

### FIELD OF INVENTION

This invention relates to a drug containing edible acid and/or acidic salt as active agent to treat and alleviate hypersensitivity diseases by lowering the humor pH; to improve individual hypersensitivity diseases by uses of drug, food, or health care food, which are made from said edible acid and/or acidic sale or the acidic fruits containing thereof, or their products; foods lowering the risk of hypersensitivity and their preparation methods; drug to lower the humor acidity and to treat or alleviate disease caused by insects bite toxin; that also to be drug for cold or virus infection; drug for inflammation; drug for analgesic; and drug for cardiovascular thrombus disease. Method lowers the hypersensitivity risk of clothing or grove and their products.

### BACKGROUND OF THE INVENTION

There are four types of disorder allergic responses to tissue damage, called hypersensitivity reactions. Type I (anaphylactic hypersensitivity, or immediate type), on account of inappropriate responses to foreign antigens relate to anaphylaxis. This anaphylaxis is IgE mediated reaction. Symptoms of IgE mediated reaction are anaphylaxis, dermatitis asthma, Parkinsonism, hay fever, and food allergy. Type II (antibody dependent cytotoxic type, or IgG and IgM mediated hypersensitivity), causes diseases such as haemolytic disease of the newborn autoimmune haemolytic anaemia, nephropathy and certain other autoimmune diseases, transfusion reactions, drug allergy and hyperacute graft rejection. Type III (immune complex mediated hypersensitivity), is mediated by IgG which causes diseases such as lupus nephritis, rheumatoid arithritis, arthus reaction, vasculitis, and serum sickness. Type IV (T cell mediated hypersensitivity, or delayed type hypersensitivity), causes diseases such as Type I hypersensitivity, chronic allergic rhinitis, contact dermatitis, tuberculin reaction, multiple sclerosis, and erythema.

Immunodeficiency is divided into inherited immunodeficiency and acquired immune deficiency syndrome, which is caused by human immunodeficiency virus (HIV). The susceptible disease for the former group includes, generally, such as respiratory infections, herpes simplex virus, chronic lung, influenza, and skin inflammatory. Drug could just block HIV replication and rise in CD4 T-cells temporary. Finally, most HIV-infected people develop acquired immune deficiency syndrome and die. However, scientists hope that will be possible to develop effective vaccines against HIV. Yet not any effective vaccine is found

Autoimmune disease is caused by a reaction of required immune system with auto antigens, which does harm to tissues. Autoimmune disease can be mediated by auto antibodies and/or by auto-active T cells. The tissue damage can be resulted from direct attack on the cells borne autoantigens, from immune-complex formation, or from local inflammation.

There are three groups of drug treating immunological disorders: first anti-inflammation drugs of the corticosteroid family, such as prednisone and antihistamine; second, cytotoxic drugs, such as azathioprine and cyclophosphamide; and third fungal and bacterial derivates, such as cyclosporine-A and rapamycin, which inhibit signaling events within T lymphocytes.

These drugs have wide action in inhibiting immune system as well as harmful ones. The beneficial effects of corticosteroids are anti-inflammation. However, there are also many side effects, including fluid retention, gain of weight, diabetes, bone mineral loss, and thinning of skin. They are caused by the results of using corticosteroids which reduces the functions of hormone and also reduces the immune functions too. The cytotoxic drug suppresses immune by killing cells. That has serious side effects, including decreasing immune function, anemia, damage to intestinal epithelium, hair loss, and fetal death or injury. The drugs of fungal and bacterial derivatives are toxic to kidney and other organs. Besides, it is expensive to ingest for a long period of treatment.

Histamine is a kind of harmful secretions in allergic reaction. That is a potent mediator in numerous biological reactions. Following the stimulation of mast cells and basophils by antigens, histamines and other compounds are released explosively into the surrounding tissues and body fluids. On releasing, histamine functions a potent mediator of numerous physiological, and causes pathophysiological processes in all organs and tissues. This reaction may result in a general depletion of vascular fluid, causing a condition known as histamine poisoning or histamine shock.

Antihistamines are used primarily to control symptoms of allergic diseases such as hay fever, arthritis and Parkinsonism. They alleviate runny nose and sneezing and, to a lesser extent, minimize conjunctivitis and breathing difficulties. Antihistamines can also alleviate itching and rash caused by food allergy. Chemically, antihistamines comprise several types. Each antihistamine neither cures all kinds of syndromes nor is good for any person. Side effects of these drugs include drowsiness, loss of concentration, and dizziness. People ingesting antihistamine should not drink alcoholic beverages or perform tasks requiring mental alertness, such as driving. Their uses in treatment are questionable. Those are the defects of traditional antihistamine.

The traditional antihistamines are compounds of amine which are high alkaline, toxic to body, damage to the stomach, and low solubility in water. For improving, the chemist applied acids, including organic acid and inorganic acid to react the amine compound to form a salt. There are many acids including inorganic acid: such as hydrogen chloride; and organic acids, such as maleic acid, citric acid, malic acid, tannic acid and succinic acid; are used.

In a diphenhydramine system, for example, the diphenhydramine is reacted with hydrogen chloride to form diphenhydramine hydrochloride; and in a chlorpheniramine system, the chlorpheniramine is reacted with hydrogen chloride to form chlorpheniramine hydrogen chloride. The other compounds such as chlorpheniramine maleate, phenyltroxamine citrate, diphenhydramine tannate, diphenhydramine salicylate, and chlorpheniramine malate are the products of reaction with organic acids of maleic acid, citric acid, tannic acid, salicylic acid and malic acid, respectively. The role of acid, such as hydrogen chloride, maleic acid, citric acid, malic acid, salicylic acid, and tannic acid, is just a modifier. That neutralizes the alkalinity of amine, lowers the amine toxicity for patients, and increases the solubility thereof at all. This is the origin of traditional antihistamine drugs which are used widely to treat allergic diseases now. Actually, there is not any antihistamine drug that shows perfect effect to allergic diseases. This made the applicant to investigate the other way of therapy and finally succeed.

Food poisoning and insect bit are two kinds of poisoning in daily life, normally. The former is caused by eating foods containing disease bacteria or toxin; and the later is caused by venom of insect bite. This toxicity could cause serious allergy reaction. The traditional treatment is to use anti-toxin and modified toxins for bacterial toxins (such as Diphtheria, tetanus toxin), and to use antivenins for insect venoms (such as black widow, snake). They are produced by vaccinating repeatedly in other animal species. Infusion a large amount of antibodies into the body will induce hypersensitivity. The disadvantage of this method is that must test in advance to make sure that the patient has not allergy history.

All the disadvantages of drugs for treating hypersensitivity disease are described hereinabove. That made the applicant to study and finish the invention.

### INVENTION CONTENT

As a result of study, the applicant found that the humor must be kept at acidic condition is necessary to performing proper biology processes. In that way, pathogens will be killed, effectively, by macrophages, by T cells and by B cells.

To describe some reasons as following:

1, In immune biology, complement is a component of plasma that tags pathogens and presents to macrophages to kill. Complement also activates T cells. The complement system is made up a large number of distinct plasma proteins that react with one another, and induce a series of inflammation responses to fight infection. Complement proteins are proteases that can be activated by proteolytic cleavage. The digestive enzyme pepsin, for example, is stored inside cells and secreted as an inactive precursor of enzyme, pepsinogen, which is only cleaved to pepsin in the acid environment (Frank, S. T., and Nealis, A. S., Immunol. Today, 12, 322˜326, 1991; Todd, J. A., and Steinman, L., Curr. Opin. Immunol. 5, 83˜89, 1993). The acidity is the necessary condition for the complement to play its role.

2, For extracellular pathogens and toxins, they are bound with MHC class II and are presented to CD4 T cells. The effect on presenting cell is the activation of B cells to secrete Ig, and to eliminate extracellular bacteria/toxins in the endocytic vesicles when the pH level is also at low (Morrison, L. A., et al., J. Exp. Med. 163, 903, 1968: Paulnock, D. M., Curr. Opin. Immunol. 4, 34˜349, 1992).

3, About 2% of oxygen can be converted into superoxide anion (.O⁻₂) during the respiration of organisms. The free radicals (FRs) of superoxide are extremely active products which can react with proteins, saccharide, fatty acid and nucleic acid FRs destroy the normal structure and disturb the normal activities of body; also cause many damages, such as cancer, cardiovascular disease, Alzheimer's disease, dementia, cataracts, Parkinsonism, immunodeficiency of old man, diabetes, inflammation, aging, and Arthritis. They all are autoimmune diseases, and are mostly induced by the damage of FRs (Harman, D., Age 7, 111-131, 1984.)

In humans, the first line of antioxidant defense is the antioxidant enzymes, especially superoxide dismutase (SOD), glutathione peroxidase (GPX). These enzymes will help destroy SOR, H2O2 and lipid peroxides.

From the chemical reaction point of view, we know that FRs, especially oxygen FRs, are mainly produced in an alkaline environment, and will be reduced by proton in an acidic condition. In this invention; the drug will provide good antioxidant to reduce the FRs.

4, There are many active peptides which are related closely to human physiological functions, such as SOD, opioid peptides (OP), immunopeptides (IP), antihypertensive peptides (AP), angiotension I-converting enzyme inhibitor (ACEI), antithrombotic peptides (ATP), and casein phosphopeptides (CPP). These peptides are formed under acidic condition, and performed their roles at the same.

The SOD, for example, catches FRs only under acidic condition. The reaction of FRs and antioxidants occur under the acidic condition, as shown in the following reactions. If not under acidic condition, the reaction can not occur to right side. And there is not any work to scavenge.

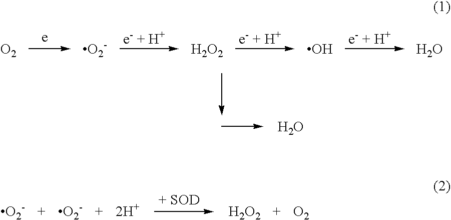

The FRs is the major factor in causing disease. If the FRs being removed, the disease so does. In treating and alleviating disease, the drugs of present invention show very widely functions, because of their excellent properties of FRs scavenging.

The necessary condition for the structure activity of ACEI is that there is a positive charge at guanidino or ξ-amino group connecting to C-end of the peptide. That proton plays a substantial role of function. As for the affinity of CCP for calcium, is caused by high level polarity of the residual group of serine phosphatide and by the stabilization effect of calcium phosphate colloid in acidic condition. It proves that how the amino acid residues affect on the physichemical action, especially, for binding ability of proton. They are determined by the acidity of solution. Therefore, the drug of this invention provides functions of preventing and depressing hypertension.

In tissue, arachidonic acid (AA) is reacted by lipoxygenase (LO) to form its derivatives, such as 12-hydroxy eicosatetraenoic acid (12-HETE) and leukotriene (LT). These products cause inflammation and hypersensitivity reactions. AA is also reacted by cycloxygenase to form prostacyclin (PGX PGI₂), thromboxanes (TXA₂), PGA₂, and PGE₂. 12-HETE activates human granulocytes. While 5-HETE is a precursor of slow reacting-substance (SRS) of hypersensitivity (Siegel M. I., et al., Proc. Natl. Acad. Sci, 77, 308-312, 1980). This indicates that there is a way to inhibit hypersensitivity and inflammation by inhibiting the reaction of LO.

In the productions of prostaglandins (PGX, PGI₂) and thromboxane A₂ (TXA₂) from AA by cyclooxygenase, we found a very close relationship between the metabolisms of lipoperoxidation and prostaglandins. This relationship leads us to find an effective antioxidant for protection. The lipoperoxidation needs a trace amount of hydrogen peroxide to initiate a reaction at the active enzyme site of Fe⁺³ of hemoglobin, and to produce oxygen FRs. The FR then gains a hydrogen atom from AA, and induces reaction. To eliminate FRs in advance, then there is no cascade to form the TXA₂ from AA. Aspirin is one of NSAIDs (non-stroid anti-inflammatory drugs). Its is proved in clinic that the reaction of aspirin inhibits the activity of cyclooxygenase, and then reduces the coagulation force of platelet. (Chau, K. Z., Oxygen free radical and clinic, 37-40, Hou Ki publisher Taipei, Taiwan, 2003).

Thrombus and embolus are produced by activated platelets. This coagulation cascade begins a series of complicated reaction one another when the damage to endothelium is happened. Releasing TXA₂, derived from AA, into plasma is the key point of clumping process, which promotes the formation of small embolus for clogging blood flow.

The drug of this invention inhibits the activity of cyclooxygenase that inhibits the cascade formation of prostaglandin, and depresses the release of TXA₂. When the formations of embolus and thrombus are inhibited, there is no way to induce cardiovascular disease, such as intracerebral and hemorrhage, and myocardia infarction. To release TXA₂ from platelets is the first message for inducing platelets to enhance the reaction of coagulation. That is the first step of clotting formation of platelet. In that case, if we could inhibit the release of prostaglandins or inhibit the activity of cyclooxygenase, we could inhibit the whole cascade of prostaglandins, and could eliminate the possible formation of thrombus, finally.

Human body has the ability to recover its natural defense in a proper condition, but will lose it when the body is weak. For recovering innate immunity, of course must strengthen the body at first. The mostly basic method is according to immunobiologic mechanisms. That is to make it sure to raise a large number of complement and to supply a good environment for the immune cells, such as marophage, CD4 T-cells, and B-ells to work. In other words, to make an acidic humor or to lower the pH of humor is necessary. That the immune mechanism could only perform in an acidic situation. This reason makes the applicant to investigate the other way of therapy. And finally, found that making an acidic situation in humor by ingesting acid compound which can enhance the ability and the functions of macrophage, CD4 T-cells and B-cells effectively. To use the acidic and edible chemicals for that purpose is the key point of this invention, and dissolve the problems of immune diseases.

The poison problems, such as food poison and insect bite, cause immune reaction in body. Drugs of this invention provide treatment in this area. The mechanism of present invention is increasing the level of acidity of humor to enhance the ability of immunity and to neutralize the toxin. Because all the toxins are proteins they could be neutralized or denatured

Saliva is a kind of humors and, normally, has the pH of around 6.8. For the purpose of reference, to test the pH of the saliva of a man was carried out who had brushed before testing and ingesting 700 mg of citric acid. Data are taken in an interval of 30 minutes for 2 hours. The results are listed as shown in table 1.

TABLE 1
The pH value of saliva is affected by acidic food

| | Testing time (minute) | | | | |
|---|---|---|---|---|---|
| | 0 | 20 | 60 | 90 | 120 |
| pH | 6.8 | 6.45 | 6.26 | 6.6 | 6.8 |

Though, the pH of saliva and urine will change in case of any acidic substance entering the body by biological mechanism. The buffer action will make the blood back to around the neutral quickly, but is at acidic side. Drugs of this invention have the function to lower the humor pH and treating or alleviating immune diseases that would be a good inflammation inhibitor.

### BRIEF SUMMARY OF THE INVENTION

Accordingly, it is an abject of the present invention to provide drugs to treat and alleviate hypersensitivity diseases.

It is provided the use of a drug for treatment or alleviating allergy disease.

The invention is to provide the uses of health care food for improving individual hypersensitivity, which is prepared with edible acid and/or acidic salt, or the acidic fruits containing thereof, and their products as active agent.

It is the principal object of this invention is to provide a method to produce lower allergy risk food.

It is therefore a general object of this invention is to provide a drug for treatment or alleviating food poison and insect toxicity disease.

It is therefore still an object of this invention is to provide a health care food for protecting or alleviating hypersensitivity diseases.

A further object of this invention is to provide an inflammation drug.

An additional object of this invention is to provide drugs for cold or virus infection, for insect bite, for treatment of cloth and it products, for thrombus and embolus, for or for analgesic.

Other and further objects of this invention will become obvious upon an understanding of the illustration embodiments about to be described or will be indicated in the appended claims, and various advantages not referred to herein will occur to one skilled in the art upon employment of the invention in practice.

### DETAILED DESCRIPTION OF THE INVENTION

This invention uses edible acid and/or acidic salt as active agent that does no harm to the body absolutely. More over, the function of this invention is to obey the most basic mechanisms of biophysics and to scavenge fire radical, but does not just one function. This invention uses edible acid and/or acidic salt as active agent that is more remarkable different from the traditional drugs, and that is the specific property of this invention.

Penicillin anaphylaxis can be avoided in using the drug of this invention. For the same reason, the death caused by vaccination also can be improved by applying the drug of this invention. Both systemic anaphylaxis and vaccination accident could be improved, by combining the ingestion drug of present invention in advance, at the same time or after their proceeding.

One benefit of this drug composition is that most of them are nature food acids and acidic salts. They can be eaten in large amount. And besides, compounding with other drug, foods and treating on foods are also possible.

The applicant found that edible acid and/or their acidic salts as active agent for the treatment and alleviation of hypersensitivity diseases by lowering pH are acids

selected from the group of comprising fumaric acid, maleic acid, malic acid, tartaric acid, citric acid, lactic acid, α-hydroxy ethanoic acid, α-octanoic acid, gluconolactone, their acidic salts of sodium and potassium, and their compounds; show wonderful effective in treating immune disease.

In the drug given by injection, we must apply small dose for the direct injecting into the tumor. The drugs of present invention have usages of oral and none oral. The proper therapeutic dose is about 0.1˜300 mg/kg/day, in generally. In special case, the ingestion dose could be much more than that according to necessary. They can be prepared in any forms of drug by the known pharmaceutics, and even combining with other active components.

Routes of drug administration of present invention may be by parenteral method, including subcutaneous, intramuscular, intravenous, intradermal, intra-arterial, intravascular, intratumor, transdermal inhalation, suppositories, ointments, aerosols, inhalants, tinctures, plasters, lotions, and mixtures. The liquid solvent includes water, alcohol, glycerin, and other glycols.

The dispensing of medication for injections is following the traditional method. To use sterilized pure water under a clean room, adjusting buffer and tonicity by sugar and salt are usually taking care of. Beside the solvent of water, ethylene glycol and polyol, such as glycerin, propylene glycol, liquid poly glycol, and mixtures are also used. Powder made by vacuum freeze-dried method is an ideal way.

The effective drugs of present invention could compound with inert dilution agent eatable carrier, sweeteners, perfumer, herbs, foods, other nutrients, and their compounds.

The oral usage of present invention could be in the forms of capsule, tablet, flake, pile, lozenges, solution, suspension liquid, syrup and blending with food.

The active agent of said edible acid and/or acidic salt is also used in foods including biscuit, cake, candy, puddings, dairies, peanut products, drinks, canned foods, cooking foods, and other processed foods. These products are coating with or containing the drug thereof. The effective agent of this invention in the product is 0.06˜10%, prefer is 0.1˜7%, better is 0.2˜4%, and the best is 0.3˜2%. (to be proved in example of table 5)

The active agent of said edible acid and/or acidic salt is also used in drinks comprising juice; wins including fruit wins, whisky, rice wins, brandy, sake, beers, herb wins; soft drinks, carbonated drinks, teas, mineral waters, alcoholic drinks sports drinks, functional drink, coffees, colas sasaparillas, dairies such as fermented milks, and herb solutions. They contain the effective agent is ranged in 0.06˜10%, prefer is 0.1˜7%, better is 0.2˜4%, and the best is 0.3˜2%. (to be proved in examples of table 5)

Edible acid and/or acidic salt of the present invention is used to treat active proteins contained in foods to form denature. The amount of drug is up to the necessary of protein contained in denaturing. It is better above the stoichiometrical quantity.

Clothing, such cloth and groove, contacting skin causing allergic reaction, can be improved by using drugs of this invention to treat the allergens and proteins contained thereof to a denature state. The skin contact allergic reactions could be inhibited.

The present invention relates to a drug containing edible acid and/or acidic salt as active agent for the treatments of anti-inflammation and anti-hypersensitivity by lowering the humor pH. Drugs of this invention are acidic compounds and just show good effect for improving and inhibiting inflammation and itchy.

By the same action of present invention, the anti-inflammation and anti-allergic reaction could be applied to inhibit and to treat diseases of cardiovascular thrombus and embolus.

In oral agents, including food and drinks, of this invention, can contain the normal components, including: binding agent; densifier, softener.disperser; emulsifier, preservative; enzyme; sweetner, perfumer, pigment; herb; another nutritiont; vegetable seed oil, cooked foods, amino acids; and their compounds.

The acidic fruits which contain the effective agent greater than 0.3%, such as plum, orange, pineapple, star fruit, grape and grape fruit could be used as drug. The content of effective agent in processed product is preference for 0.3% than 0.06%.

As an oral drug, when the effective agent compounding with food, the dose would be changed depending on the amount of food ingested. In a low concentration of effective agent, food must ingest a greater amount rather than a higher concentration one. Taking 300 mg/dose, for example, a man ingests 500 ml or 500 gr of food once a time when the food must contain 0.06% of effective agent. The normal quantity of drink is about 250 ml or 250 gr, when the same agent of 300 mg/dose in it is 0.12%. But the patient ingests drug with water is about 100 ml or 100 gr a time, when the concentration of drug in food is 0.3%. By this relationship described hereinabove, the content of drug of acids and/or acidic salt is 0.06˜10%, prefer is 0.1˜100%, the better is 0.2˜100%, and the best is 0.3˜100% (to be proved in example of table 4).

Therefore, the amount of edible acid and/or acidic salt contained in foods, dishes, drinks or health care products is 0.06˜100%, prefer is 0.1˜100%, the better is 0.2˜100%, and the best is 0.3˜100% (based on the total weight of food, drink or health care product in wt/wt).

Present invention, accordingly, edible acid and/or acidic salt could be used to treat food.

An allergic food is a food containing active proteins which could cause allergy disease. That could be inhibited by denaturing the active protein. The agent of present invention is the best one to denature the allergic protein. The concentration of edible acid and/or acidic salt is 0.06˜10%, prefer is 0.1˜7%, the better is 0.2˜4%, and the best is 0.3˜2%.

For food allergy-sensitive person, sea foods, especially crab and shrimp, are very potent allergens. There is one way to prevent the immune disease from eating those foods. To add a proper amount of effective component of present invention in processing sea food is very suitable. The product of such treated sea food not only can avoid the allergy reaction, but also prevent the unsaturated fish oil from oxidation because of the antioxidant reaction of effective component.

The efficient of present invention drugs for immune disease is proportion to the number of carboxyl group contained in the same compound. The citrate compounds, for instance, the power series is as following:
Citric acid>dihydrogen citrate>monohydrogen citrate

In this invention the individual means any spondyle animal, the better is mammal, and the best is human.

### EXAMPLE

This invention will be understood more readily with reference to the following examples. These examples, however, are intended to illustrate the invention and are not meant to limit the scope of the invention.

### Example 1˜10

### Anti-Allergy Reaction

This is a comparative testing of drug depressing effect on the amount of leaching histamines when is treated with 48/80 (Sigma, St. MO, USA) compound.

(1). Preparation of leaching cell solution from mouse body.

A mouse is killed and bloodletting. Then 10 ml of Locke's solution containing 0.1% bovine serum protein is injected into its abdominal cavity. After abdominal cavity being light massaged, the cavity is cut and the Locke's solution is removed. Cavity is washed with another 5 ml of Locke's solution, and this washed solution is added to the late one. This combined solution is centrifuged at 600 rpm for 5 minutes. The sediments are washed with 5 ml of cool Locke's solution. Adding 3 ml of cold Locke's solution to the washed sediments, then a leached cell solution of abdominal cavity is obtained. The composition of Locke's solution is: NaCL 9.1%, KCL 0.2%, CaCL₂ 0.15%, glucose 1.0%, in w/v, and the rest distillated water.

(2). Drug depressing effect on the amount of leaching histamines when treated with 48/80 compound.

Each testing compound listed in the table 1 is dissolved in a Ringer's solution containing 1% NaHCO₃, and then diluted with Locke's solution to the indicated concentration. 1.0 ml of each of those solutions in last term is mixed with 0.3 ml of mouse's leaching cell solution and 0.5 ml of Locke's solution. This mixture is cultivated at 37° C. for 5 minutes. Then adding 0.2 ml of Locke's solution of 48/80 compound (1 mg/100 ml) and cultivated at 37° C. for 10 minutes. Then the reaction is stopped by cooling, and centrifuged at 2,500 rpm for 10 minutes. 1.7 ml of decanted solution and 0.3 ml of sediments are obtained. 0.1 ml of water and 0.2 ml of 100% trichloroacetic acid are added to the decanted solution. 1.5 ml of Locke's solution and 0.2 ml of 100% trichloroacetic acid are added to the sediments washed solution. They are cultivated at room temperature for 30 minutes. After cultivation, the mixtures are centrifuged at 3,000 rpm for 15 minutes, respectively. 0.35 ml of each of the former two solutions is sampled. In each sample, 1.65 ml of water, 0.4 ml of 1N NaOH and 0.1 ml of 0.5% OPT (o-phthalic aldehyde) in methanol are added and cultivated at room temperature for 4 minutes. The reaction is stopped by adding 0.2 ml of 2M citric acid. And finally, determine the amount of released histamines in the tested solution by fluorescence method. By the analysis, results of the depression rate of histamines could be calculated.

Locke's solution is instead of each drug in control group, and instead of both drug and 48/80 compound solution in blank group. The histamine releasing rate (A) can be calculated by following equation. Where (Hs) is the total amount of histamine in the decanted solution, and (Hr) is the total amount of histamine in the sediment. (A)=[(Hs)/{(Hs)+(Hr)}]×100%. Then the depression rate is:
=100−[(A−A in blank group)/(A−A in control group)]×100%.

The calculated results are shown as following table 2.

TABLE 2
Drug depressing effect.

| | | Histamine | |
|---|---|---|---|
| Testing | Testing drug | Releasing | Depression rate |
| No. | 100(mg/ml) | rate (%) | (%) |
| Control | Control | 90.5 | — |
| group | | | |
| Blank | Blank | 9.0 | — |
| group | | | |
| (1) | Trisodium glycyrrhizinate | 65.5 | 30.9 |
| (2) | Diphenhydramine hydrochloride | 64.7 | 32.1 |
| (3) | Citric acid | 8.7 | 100 |
| (4) | Lactic acid | 8.9 | 100 |
| (5) | Malic acid | 9.0 | 100 |
| (6) | Tartaric acid | 8.9 | 100 |
| (7) | Fumaric acid | 8.9 | 100 |
| (8) | α-hydroxy ethanoic acid | 9.0 | 100 |
| (9) | α-hydroxy octanoic acid | 9.0 | 100 |
| (10) | gluconolactone | 8.9 | 100 |

Trisodium glycyrrhizinate and diphenhydramine hydrochloride are traditional antihistamines. It is quite obvious that the results of drugs of present invention show completely affective while the traditional drugs are incompletely.

The inhibiting effect of histamine can also inhibit the production of compounds, such as 12-HETE, LT, PGX PGI₂, TXA₂, PGA₂ and PGE₂, of course, there is no thrombus disease happened.

### Example 11˜20

### Anti-Delayed Type Allergy Reaction

The weights of testing mice are ranging from 20 g to 30 g. They are coated with 0.1 ml of oxazolone alcohol solution (0.5 w/v %) on the hair cleaned part of abdomen. After five days, each of the listed drugs is dissolved in oxazolone acetone solution (0.5 w/v %), and 10 μl each of the solutions is token by micro pipette to coat on both sides of the right ear. After 24 hr, the mouse is killed by ether and punched a circle area of a diameter of 5.5 mm on both right and left ears in corresponding part by a puncher machine (portions of drug coated and the blank). The punched portions are weighed and the inflammation rates calculated. The control group are coated only with the oxazolone acetone solution (0.5 w/v %). The inflammation depressing rates of each drug are calculated by following equation:
Inflammation depressing rate(%)=[(weight of drug coated right ear)−(weight of non-drug-coated left ear)]×100%/(weight of non-drug-coated left ear)

The inflammation depressing rate of each drug is shown in table 3.

TABLE 3

| | | Drug-coated | | Depression |
|---|---|---|---|---|
| Testing | | Amount | Mouse | rate |
| No. | Testing drug | (mg/ear) | (number) | (%) |
| (11) | Diphenhydramine | 1 | 6 | 20 |
| | hydrochloride | | | |
| (12) | Citric acid | 1 | 7 | 100 |
| (13) | Lactic acid | 1 | 6 | 97 |
| (14) | Malic acid | 1 | 7 | 96 |
| (15) | Tartaric acid | 1 | 7 | 97 |
| (16) | Fumaric acid | 1 | 6 | 98 |
| (17) | α-hydroxy ethanoic | 1 | 6 | 98 |
| | acid | | | |
| (17) | α-hydroxy octanoic | 1 | 6 | 94 |
| | acid | | | |
| (19) | gluconolactone | 1 | 7 | 97 |

Table 3 shows that the anti-inflammation rates of traditional anti-histamine drugs are very poor in comparison with this invention. Drug could be anti-inflammation, is also could be analgesic.

### Example 20

### Testing in Sea Food Eating

An adult man who is very serious allergic to sea foods, especially shrimps, he ingests two capsules of this invention drug (1,000 mg, 30 wt % of garlic and 70 wt/% of citric acid) before eating shrimps. After ate many shrimps there is not any symptom of allergy at all.

The same people before eats crabs dishes have ingested tow capsules of traditional strong anti-histamine (containing trisodium glycyrrhizinate 108 mg, oratic acid 60 mg, chlorpheniramine 5 mg, Ta Fong Co,). No sooner he has ate, he feels bad in tasting and fells sick very bad when he have been sent to hospital for treatment.

### Example 21˜26

### Treating Cold

The oral dose of this invention such as tablet and capsule can increase the number of tablet or capsule. The following examples will explain how the effective agent of drug is required in a food of 100 ml volume.

There are six different doses (10 mg, 60 mg, 100 mg, 300 mg, and 600 mg of malic acid) of testing solution, which content the basic compounds of water 100 ml, propylene glycol alginate 0.1 g, fructose 10 g, garlic 300 g, zinger 100 g, angelica ainensis radix 10 mg, honey 3 g, armeniacae semen 10 mg. These six drugs are given six groups, 5 patients per group, of catching cold patients individually per tow hours a time. The ingestion of drug is stopped when the cold syndromes are improved. The effect of drugs by treating time is listed in Table 4.

TABLE 4
Time and dose for treating cold

| | Example | | | | | |
|---|---|---|---|---|---|---|
| | 21 | 22 | 23 | 24 | 25 | 26 |
| Dose, mg | 10 | 60 | 100 | 200 | 300 | 600 |
| Rate of malic acid | 0.01 | 0.06 | 0.10 | 0.2 | 0.3 | 0.6 |
| in foods, % | | | | | | |
| Time for | 8 | 4.5 | 3.2 | 2.2 | 1.6 | 1.1 |
| treatment*, day | | | | | | |
| ranking | Poor | good | better | best | excellent | excellent |

*the time for treatment needed is the average of the same group.

Therefore, the content of this invention in dose must be expressed in 0.06˜100%, good is in 0.1˜100%, better is in 0.2˜100% and the best is in 0.3˜100%. Normally, the higher the concentration, the little amount of foods could be taken is. In the medicament there is a regulation of mg/day/kg for the toxic drug, while this invention is belonging to foods and it to be better expressed by concentration in food ingestion one time.

### Example 27˜37

### Testing the Upper Limit Concentration of Drug for Taste of Drug Containing Foods

When applies the this invention to foods and lowering the risk of allergy or health care foods, a higher drug content is better for the disease, but a taste problem will be faced. There is needed to limit the drug content. Taste testing is carried out as follows.

To make WU LOONG tea by 250 g of tea with 8.3 liters of 80□water and 420 g of sugar is added. Different doses of 1 g, 2 g, 3 g, 4 g, 5 g, 6 g, 7 g, 8 g, 9 g, 10 g and 12 g of malic acid are added into each set of 11 cups of 100 ml Wu LOONG tea individually. Let 6 volunteers to test the 11 cups of tea individually. Each person ranks the taste of tea by five levels of best, better, good and acceptable and can not acceptable. The results are shown in Table 5.

TABLE 5
The results of taste testing

| | Amount of | | | | |
|---|---|---|---|---|---|
| Example | malic acid, % | Best | Better | Good | acceptable |
| 27 | 1.0 | 6 | | | |
| 28 | 2.0 | 6 | | | |
| 29 | 3.0 | 2 | 4 | | |
| 30 | 4.0 | | 6 | | |
| 31 | 5.0 | | 3 | 3 | |
| 32 | 6.0 | | 1 | 5 | |
| 33 | 7.0 | | | 6 | |
| 34 | 8.0 | | | 4 | 2 |
| 35 | 9.0 | | | 2 | 4 |
| 36 | 1.0 | | | | 6 |
| 37 | 12 | | | | not acceptable |

The results shows that the levels of acceptance are: the upper limit of acceptable concentration is <10%, good is <7%, better is <4%, best is <2% and not acceptable is 12%. To combine the test data of lower effective limit concentration in example 52, the concentration of drug in foods must be: normally is 0.06˜10%, good is in 0.1˜7%, better is in 0.2˜4% and the best is in 0.3˜2%.

### Example 38

### Oral Syrup of Orange Skin

The formulation comprises 50 ml (62% alcohol) of orange skin tincture, 50 g of citric acid, 15 g of talc powder, 850 g of sugar and the balance is distillated water to make 1000 ml. This mixture is filtrated, sterilized and bottled as product.

### Example 39

### Injection

To dissolve 36 g of citric acid and 34 g of potassium dihydrogen citrate in a total volume of 1000 ml of sterilized water, then the solution is sucking filtrated through a ceramic filter, and filled in 10 ml ample by normal GMP procedure in a clean room.

### Example 40

### Capsule

Grinding and compounding 350 g of citric acid, 200 g of garlic powder, 50 g of zinger powder, 10 g of angelica sinens radix powder, 10 g of armeniacae semen powder and 300 g of fructose, and the compound is encapsulated to 1000 pieces of product.

### Example 41

### Granular and Tablet

The formulation comprises 30 g of maleic acid, 20 g of corn starch, 20 g of lactose, 5 g of Ca-CMC, 5 g of polyethylene pyrrolidone, and 10 g of talc. To grind maleic acid, corn starch and lactose to fine powder, then the compound is produced in a product of 1˜2 m/m granular by normal granular machine, using 5% water solution of poly ethylene pyrrolidone as a binder.

To mix talc and the produced granular, and then product of 100 tablets of containing 300 mg maleic acid are produced by tablet machine.

### Example 42

### Powder

The formulation comprises 50 g of fumaric acid, 400 g of microcrystalline cellulose and 550 g of corn starch. To dissolve the fumaric acid with 200 ml of pure water and being adsorbed by microcrystalline cellulose, the product is dried and then mixed with corn starch to form a twenty folds powder.

### Example 43

### Pills

The formulation comprises 50 g of succinic acid, 1 g of potassium dihydrogen phosphate, 50 g of glycyrrhizin, 5 mg of ginseng, 1 g of zingier, 5 g of starch and 50 g of honey. To pulverize succinic acid first an then compounding with other components by a kneader, and finally a product of 150 pills containing 320 mg of succinic acid per pill are produced by a pilling machine.

### Example 44

### Troches

The formulation comprises 100 g of α-hydroxy octanoic acid, 80 g of gelatin, 200 g of glycerin, 20 g of acacia gum, and 160 g of perfume water. To pulverize the α-hydroxy octanoic acid into powder first and is added to the transparent solution that is prepared by following steps. Gelatin and acacia gum are softened with proper amount of water, then glycol is added and heated to form a transparent solution. Into this solution the powdered α-hydroxy octanoic acid is added and mixed gently, poured into a mold and cooled to as products.

### Example 45

### Emulsions

The formulation comprises 100 g of succinic acid, 20 g of span-60, 100 mg of ethyl p-hydroxy benzoate, and the balance amount of peanut oil. Pulverizing the mixture of succinic acid and span-60 by grinding machine, then ethyl p-hydroxy benzoate and peanut oil to make a total volume of 1000 ml are added and mixed strongly for three minutes, and bottled as products.

### Example 46

### Blending Foods (Canned Fish Foods)

10 kg of sardines are washed. After their heads and tails being cut and the inner organics being cleaned up, they are cut into a proper size. These raw materials are cooked in a 201 solution that contains 1.2 kg of salt and 800 kg of citric acid. The cooked fish then is canned into No. 4 size steel can with 75 g of tomato ketchup, and the product then is sterilized by normal process.

### Example 47

### Blending in Foods (Cookies)

The formulation comprises 10 kg of wheat powder, 3.5 kg of sugar, 0.8 kg of shortening oil, 1 kg of millet jelly, 0.03 kg of salt, 0.2 kg of ferment, and 0.62 kg of α-hydroxy ethanoic acid. To follow the traditional method of cake making, the solids of wheat powder, sugar, salt, and α-hydroxy ethanoic acid are ground and sieved individually first. They are mixed them with ferment and part of wheat powder, and compounded well with millet jelly and shortening oil. After shaping, and baking in two stages; first stage is at 180˜2000□, and the second stages is at 150˜205□; products are produced.

### Example 48

### Blending in Foods (Cakes)

The formulation comprises 1 kg of wheat powder, 1 kg of sugar, 1 kg of egg, 150 g of gluconolactone, and 300 g of water. The albumin and egg-yellow are separated first and the former is bubbled by bating. After the albumin is bubbled, sugar, gluconolactone, and water are added and mixed homogeneously. The wheat powder is sieved and added to the mixture. To mix quietly and being molded for baking, then cakes are produced.

### Example 49

### Blending in Foods (Peanut Products)

The formulation comprises 1 kg of peanut, 20 g of salt, 25 g of fumaric acid, 50 g of lecithin, 20 mg of pineapple enzyme and 2 ml of ethanol. The peanut is roasted at 160□ for 1 hour and ground into powder after drying, and sieved to remove the skins and germs. To add salt, lecithin, pineapple enzyme (which is dissolved in alcohol first), and fumaric acid consequently, and is ground to form paste before packing in a 500 g bottle.

### Example 50˜59

### Health Care Food Made of Fruits

Fruits which contains at least 0.3% of the effective component of this invention, such as acidic orange, lemon, plum, fruit orange, grape, apple, carambola, strawberry, and pineapple are processed to produced cans by normal method including: selecting, clearing, removing stalks, cutting heads and tails, skin and core removing, buds removing, slicing, canning, weighing, syrup adding, sterilizing, cooling, inspection, and packing.

The former examples use pure chemicals as effective component. Now, to use acid contained fruits replace the pure chemicals in these examples. For the low acid contained fruits, the juice must be concentrated to increase the acids level, and then to take the place of pure chemicals in these examples.

Taking orange juice, for instance, the juice is compounded with 5 kg of orange juice containing acidity 1.0% and 150 g of citric acid. There is 200 g of citric acid in a 10 l of orange juice drinks. How much quantity of fruits is needed when producing the same 10 l juice with different levels of citric acid containing, the results are shown as table 6.

TABLE 6
The equivalent dose of effective component in using fruits

| | | | The amount of fruit | |
|---|---|---|---|---|
| | | Acidity, | equivalent to 200 g | |
| Example | Fruit | % | citric acid, kg | notation |
| 50 | Orange (1) | 6.0 | 3.33 | |
| 51 | Orange (2) | 4.0 | 5 | |
| 52 | Lemon | 7.0 | 2.58 | |
| 53 | Plum | 3.8 | 52 | |
| 54 | Grape fruit | 2.0 | 10 | |
| 55 | Grape | 1.0 | 20 | Needed |
| | | | | concentration |
| 56 | Apple | 0.5 | 40 | Needed |
| | | | | concentration |
| 57 | Carambola | 5.0 | 4 | |
| 58 | Straw berry | 0.8 | 25 | Needed |
| | | | | concentration |
| 59 | Pineapple | 4.5 | 4.4 | |

Examples of 55, 56 and 58 have volume more than 10 l, they are needed to concentrate in order to get their acidity greater than 1.0.

The edible organic acids are the effective component of this invention, so that to use the organic acid contained fruits are reasonable. The other compounds of fruits are not important just as pharmaceutical acceptable carriers.

### Example 60

### Coffee (Instant Coffee and Packed Coffee Solution)

The formulation comprises 10 kg of coffee bean, 1.5 kg of malic acid, 9.6 kg of sugar, 7.2 kg of cream, and water for balance.

Coffee beans are roasted, ground and heat water extracted under pressure, and a 30% coffee of 10 l solution is obtained. The malic acid is added into the resulted solution. The solution is concentrated by the frozen method and frozen dried under nitrogen gas. A 4.5 kg of instant coffee product containing 33% of malic acid is produced.

That coffee product is further compounded with 9.6 kg of sugar and 7.2 kg of cream, and packed in a 17 g content product of carry-pack instant coffee.

A kind of liquid coffee drinks are made from the 30% coffee contained solution. That is compounding with 1.5 kg of malic acid, 9.6 kg of sugar, 7.2 kg of cream and the balance of water to make up of 240 liters. After heating and cooling, to pack in a volume of 200 ml, then 1200 packs of liquid coffee are produced.

### Example 61˜65

### Tincture and Treatment for Inflammation, Analgesic and Itchy

The formulation comprises 10 g of citric acid, 5 g of glycerin, and 90 ml of alcohol (70 v/v) in a mixture.

A series of testing are carried out by a group of five patients for each syndromes, which treating the topical disease three times a day, the results is shown as table 7.

TABLE 7
Results of treating inflammation, analgesic and itchy

| Example | Diseases | Treating results |
|---|---|---|
| 61 | Acne(pain) | One day scaled, pain improved |
| 62 | Insect bite (itchy, | Itchy disappeared in half hour, |
| | inflammation, pain) | inflammation disappeared after |
| | | 3 hours, and pain improved |
| 63 | Pruigo(itchy) | One day improved |
| 64 | Skin wound(pain) | After dried the wound released, pain |
| | | improved, healing quickly |
| 65 | Pustules (pain) | The pustules shrunk one day, scaled |
| | | after 2 days, pain improved |

### Example 66

### Improving the Allergic Risk of Dairy Products

The formulation comprises 10 l of milk, 10 g of citric acid and 3 g of Ca-CMC.

During mixing milk, Ca-CMC is added in homogenous and then citric acid is added to produce a none-allergic dairy milk. The product may be produced into milk powder by spray-drying machine.

### Example 67

### Shrimps Processing

The formulation comprises 10 kg of litter shrimps, 360 g of salt, 360 g of citric acid, and 20 l of water.

The shrimps are set in a basket and washed in a following water to remove sand. The washed shrimps are treated in a 20 l boiling water, containing salt and citric acid, for 25 minutes. The boiled shrimps are sun-drying on straw mats out door. The dried shrimps are packed in 250 g. The treated products are good for reserving and allergy risk free for allergic persons.

### Example 68

### Salt Fish (Little Sardine)

The formulation comprises 10 kg of little sardine, 1.2 kg of salt, 1 kg of citric acid, and 20 l of water.

The sardines are washed in a trough and spread on a ten-layer boiling cage, and then treated in a volume of 20 l boiling solution in kettle, containing salt and citric acid, for a period of time until the solution boiling again. Before products are removed from the kettle, the upper layer oily floats are washed away by adding new solution. The boiled fishes are sun-dried with the cage turning the other side ever day. In summer day, they can be dried in about 3 days.

### Example 69

### Production of Allergy Free Medical Groove

The formulation comprises 200 parts of natural rubber latex (pH10.5, containing ammonia, solid component 50%, Taiping Perak, Malaysia), 75 parts of boric acid treated casein (solid component 10%), 10.0 parts of zinc oxide dispersion solution (solid component 50%), 133 parts of corn starch slurry crossing treated by epichlorohydrin (solid component 50%), 1 part of sulfur powder, 0.05 parts of carboxyl polymethylene polymer (molecule weight 500,00˜1,000,000), the rest is deionized water to dilute to form a 10% solid containing. The coacervation agent is 45% calcium nitrate in deionized water.

The production is according to the conventional method: hand model dipping into latex solution, drying, dipping into latex solution, drying, dipping into latex solution, drying, folding edge, drying, crosslinking treatment, washing, drying, dipping into 5% citric acid solution, drying, powdering (5˜40 μMgO), removing from model, and finally, packing for product.

If do not us dipping with liquid solution of the effective agent of present invention, the effective agent could be pulverized to form a sized of 5˜4μ and mixing with MgO in a rate of 4% for powdering. Testing is carried out for five medical persons who have allergic reactions to latex grove. The results shown none had the allergic reaction.

### Example 70

### Glucose Injection (Containing Other Active Agent)

To dissolve 500 g of glucose and 10 g of citric acid in 10 l of high pressure sterilized water in a clean room. The solution is filtered by ceramic filter and packing into a 500 ml injection product by the GMP method.

### Example 71

### Testing for Anti-Free Radicals

The determination of free radical content is performed by using individual free radical testing kit, of BioVitale Inc. (Irvine, La., USA). The process is as follows: to take the specific amount of urine by pipette, open the testing agent ampoule and adding the urine, shaking the ampoule for 5 minutes, comparing the color of ampoule solution with these colors listed in table of kit. The table of free radical content level is divided into 4 classes: most proper level (0), low level (+1), medium level (+2), and high level (+3).

The volunteers selected 5 persons. In the group, 2 persons have medium free radical levels in urine, and the rest are high levels. They are given the agent as shown in example 51 three times a day individual, and sampling the urines 2 day after. The results are shown in Table 8. The effective agent of this invention shows good in anti-free radical.

TABLE 8
free radical content in urine

| | | Free radical |
|---|---|---|
| | Free radical | content in urine |
| | content in urine | after administrated |
| Item | before testing | the drug of this invention |
| Person 1 | +2 | 0 |
| Person 2 | +2 | 0 |
| Person 3 | +3 | 0 |
| Person 4 | +3 | 0 |
| Person 5 | +3 | 0 |

### Example 72˜76

### Testing for the Depression of Enzyme Activity

We recognized that the effective agent of present invention shows the ability of treating histamine, inflammation, analgesic, and achy from examples of 1˜10 and 61˜65. It goes without saying that they also could depression the cascade of the production of prostaglandins and other chemicals. Therefore, there are not any embolus and thrombus to be happened, and could avoid cardiovascular diseases such as congestion of the brain and myocardial infarction. The first step of formation for a clot is that the releasing of thromboxane from platelet induces the message of enhancing coagulation. The free radical of peroxide is a major factor for activating cyclooxygenase in prostaglandin cascade. It is clear from the testing results of example 98, when the drug of this invention existing, that cascade could not be happened, because the free radical is inhibited by the drug of this invention.

The production process of peroxide free radical can be tested by Xanthine oxidase substrate in vito (Fridowich, I., J. Biol. Chem., 215, 4053˜4057, 1970). This method is applied to prove the effect of this invention. The test of inhibiting xanthine oxidase is carried out by the method of H. M. Kalckar (J. Biol. Chem., 167, 429˜443, 1947). The basic principle is xanthine being acted by xanthine oxidase to form uric acid that is quantitative analyzed by photometric method. By the amount of determined uric acid, is calculated the degree of inhibiting effect for the activity of xanthine oxidase.

The testing process is, adding a final concentration of 0.01 u/ml of xanthine oxidase in 1 cm cell of photometric apparatus, and adding 0.05M (pH=7.4) of phosphoric acid buffer or inhabitant The reaction time is counted from the point at adding xanthene's to a final concentration reaching 5×10⁻⁵M. For reducing the error arose by absorption of liquid in photometric analysis, the compound of xanthine oxidase, xanthine and drug are boiled for the reference. UV selected at 295 nm, data are recorded at an interval of 30 seconds for 2 minutes. The unit of activity change of xanthine oxidase is 0.001M/min. Calculate the inhibiting rate for each addition amount of drug. To use drug concentration (M) as a function of inhibiting rate (%), and to trace the data on a log scale paper, the 50% depression rate of the oxidase (IC50) could be determined by regression line method.

The drugs tested are citric acid, malic acid, tartaric acid and fumaric acid, and using folic acid for comparison. The results are listed as table 9. The drugs of this invention show high inhibition effect.

TABLE 9
Testing results for IC50

| | | IC50 (concentration |
|---|---|---|
| | | for depression of 50% |
| Example | Drug | activity of oxidase) |
| 72 | Citric acid | 1.00 × 10⁻⁷ M |
| 73 | Malic acid | 1.12 × 10⁻⁷ M |
| 74 | Tartaric acid | 1.02 × 10⁻⁷ M |
| 75 | Fumaric acid | 1.01 × 10⁻⁷ M |
| 76 | Folic acid | 6.62 × 10⁻⁷ M |

### Example 77

### Testing for Analgesic

The formulation of drug for testing comprises 300 mg of malic acid, 300 mg of tartaric acid, 300 mg of citric acid, 50 mg of caffeine and 10 mg of catechin. Five volunteers of head-ache and physical pain are given one dose individual. To record the reaction after treatment, they all felt their pains are improved in 10˜30 minutes.

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. A drug composition containing the effective components of edible acid and/or acidic salt and any pharmaceutical acceptable compound to lower the humor pH and treating or alleviating hypersensitivity diseases.

26. A drug composition according to claim 1 wherein said edible acid and/or their acidic salt is selected from the group of comprising fumaric acid, maleic acid, malic acid, tartaric acid, citric acid, lactic acid, α-hydroxy ethanic acid, α-octanoic acid, gluconolactone, their acidic salts of sodium and potassium, and their compounds.

27. A drug composition according to claim 1 wherein said hypersensitivity disease is selected from the group comprising allergy, immunodeficiency and autoimmunity.

28. A drug composition according to claim 1 wherein said edible acid and/or their acidic salt concentration is 0.06˜100(wt/wt) %, prefer is 0.1˜100(wt/wt) %, better is 0.2˜100(wt/wt) %, and the best is 0.3˜100(wt/wt) %.

29. A drug composition according to claim 1 wherein said drug is selected from group comprising oral agent, non-oral agent, or topical usage.

30. A drug composition according to claim 1 wherein said drug is oral agent selected from the group comprising capsule, tablet, flake, granule, powder, pile, lozenges, syrup, beverages, solution, suspension liquid, and blending in food.

31. A drug composition according to claim 1 wherein said drug is an oral agent, compounding with compounds selected from group comprising: binder agent; densifier; softener; disperser; emulsifier; preservative; lubricating agent; enzyme; sweetener; perfume agent; pigment; herb; another nutrient; and their compounds.

32. A drug composition according to claim 1 wherein said drug is injection selected from the group comprising intradermal injection, muscles injection, venous injection, hypodermic injection, the join lubricating liquid, intestine and in tumor.

33. A drug composition according to claim 1 wherein said drug is none oral agents such as liquid, paste, and skin absorption; in which the solvent is selected from the group comprising water, alcohol.

34. A health care food containing the effective components of edible acid and/or acidic salt and any food acceptable compound to lower the humor pH and preventing or alleviating hypersensitivity diseases.

35. A health care food according to claim 10 wherein said wherein said edible acid and/or their acidic salt is selected from the group of comprising fumaric acid, maleic acid, malic acid, tartaric acid, citric acid, lactic acid, α-hydroxy ethanic acid, α-octanoic acid, gluconolactone, their acidic salts of sodium and potassium, and their compounds.

36. A health care food according to claim 10 wherein said food is selected from the group comprising biscuits, cake, candy, canned foods, diary product, peanut product, pudding, egg product, cooking dishes.

37. A health care food according to claim 10 wherein said food is selected from the group comprising juice, wine, (such as fruit wine, whisky, brandy, sake, beer, and herb wine) soft drink, carbonated drink, none carbonated drink, tea, sports drink, functional drink, coffee, cola, sarsaparilla, diary product such as fermented milk, and herb drink.

38. A health care food according to claim 10 wherein said edible acid and/or acidic salt is 0.06˜10(wt/wt)%, prefer is 0.1˜7(wt/wt) %, better is 0.2˜4(wt/wt)%, best is 0.3˜2(wt/wt)%.

39. A health care food according to claim 10 wherein said edible acid and/or acidic salt is contained thereof more than 0.06(wt/wt)%, prefer more than 0.3(wt/wt)% in a form of processed fruit selected from the group comprising acidic orange, lemon, plum, fruit orange, grape, apple, carambola, strawberry, and pineapple.

40. The use of the effective components of edible acid and/or acidic salt and any pharmaceutical acceptable compound to lower the humor pH value and treating or alleviating hypersensitivity diseases in a drug composition.

41. The use of the effective components according to claim 16 wherein said edible acid and/or their acidic salt is selected from the group of comprising fumaric acid, maleic acid, malic acid, tartaric acid, citric acid, lactic acid, α-hydroxy ethanic acid, α-octanoic acid, gluconolactone, their acidic salts of sodium and potassium, and their compounds.

42. The use of the effective components of edible acid and/or acidic salt and any food acceptable compound to lower the humor pH value, and to prevent and improve individual allergy disease is used in health care products.

43. The use of the effective components according to claim 18 wherein said wherein said edible acid and/or their acidic salt is selected from the group of comprising fumaric acid, maleic acid, malic acid, tartaric acid, citric acid, lactic acid, α-hydroxy ethanic acid, α-octanoic acid, gluconolactone, their acidic salts of sodium and potassium, and their compounds.

44. The use of the effective components according to claim 18 wherein said edible acid and/or acidic salt is an acidic fruit containing thereof selected from the group comprising acidic orange, lemon, plum, fruit orange, grape, apple, carambola, strawberry, and pineapple.

45. The use of the effective components according to claim 18 wherein said foods is oral agent compounding with food selected from the group comprising cake, biscuits, candy, canned foods, diary products, peanut products, pudding, egg products, cooking dishes.

46. The use of the effective components according to claim 18 wherein said drink is selected from the group comprising juice; wine, such as fruit wine, whisky, brandy, sake, beer and herb wine; soft drink, carbonated drink, none carbonated drink, tea, sports drink, functional drink, coffee, cola, sarsaparilla, diary product such as fermented milk, and herb drink.

47. A method of preparation that lowers the risk of food allergy caused by active proteins comprises treating the said food with edible acid and/or their acidic salts solution.

48. The method according to claim 23 wherein the said active protein containing one food is selected from the group comprising fish, shrimp, crab, butter peanuts, milk, or powder milk.

49. The method according to claim 23 wherein said edible acid and/or their acidic salt is selected from the group of comprising fumaric acid, maleic acid, malic acid, tartaric acid, citric acid, lactic acid, α-hydroxy ethanic acid, α-octanoic acid, gluconolactone, their acidic salts of sodium and potassium, and their compounds.

50. The method according to claim 23 wherein the concentration of said edible acid and/or acidic salt is 0.06˜10(wt/wt)%, prefer is 0.1˜7(wt/wt) %, better is 0.2˜4(wt/wt)%, best is 0.3˜2(wt/wt)%.

51. A method for producing allergy free clothing that is treated with edible acid and/or their acidic salts.

52. A method for allergy free clothing according to claim 27 the said clothing is groove or clothes.

53. The method according to claim 27 wherein said edible acid and/or their acidic salt is selected from the group of comprising fumaric acid, maleic acid, malic acid, tartaric acid, citric acid, lactic acid, α-hydroxy ethanoic acid, α-octanoic acid, gluconolactone, their acidic salts of sodium and potassium, and their compounds.

54. The method according to claim 27 wherein the concentration of said edible acid and/or acidic salt is 0.06˜10(wt/wt)%, prefer is 0.1˜7(wt/wt) %, better is 0.2˜4(wt/wt)%, best is 0.3˜2(wt/wt)%.

55. The products of any claims as claimed 20˜30.

56. A drug composition according to claim 1 wherein said drug is an anti-inflammatory drug.

57. A drug composition according to claim 1 wherein said drug is cold and antivirus drug.

58. A drug composition according to claim 1 wherein said drug is drug of treating toxin.

59. A drug composition according to claim 1 wherein said drug contains another effective compound.

60. A drug composition according to claim 1 herein said drug is agent for prevention and treating cardiovascular thrombus disease.

61. A drug composition according to claim 1 wherein said drug analgesics.

62. A drug composition according to claim 1 wherein said drug is for spondyle animal, prefer is for mammal, and the best is for human.

63. A method that uses the effective components of edible acid and/or acidic salt to lower the humor pH and to treat or to alleviate hypersensitivity diseases.
